(19) United States
(12) Patent Application Publication
(10) Pub. No.: US 2024/0145065 A1
(43) Pub. Date: May 2, 2024

(54) APPARATUSES, SYSTEMS, AND METHODS FOR A REAL TIME BIOADAPTIVE STIMULUS ENVIRONMENT

(71) Applicants: Robert DOUGHERTY, Redwood City, CA (US); Compass Pathfinder Limited, Altrincham, Cheshire (GB)

(72) Inventors: Robert F. Dougherty, Redwood City, CA (US); Joanna Kuc, London (GB); Ekaterina Malievskaia, London (GB); Gregory A. Ryslik, Westerville, OH (US)

(21) Appl. No.: 17/927,468

(22) PCT Filed: Nov. 22, 2022
(86) PCT No.: PCT/US22/50755

§ 371 Date: Jun. 1, 2023

Related U.S. Application Data
(60) Provisional application No. 63/282,635, filed on Nov. 23, 2021.

(51) Int. Cl. G16H 20/70; G06F 3/01; G06T 13/40

(52) U.S. Cl. CPC G16H 20/70; G06F 3/011; G06T 13/40

(57) ABSTRACT

Approaches for providing a real time bioadaptive stimulus environment with audio, visual, and olfactory components to enhance psychedelic therapy are provided. A virtual bioadaptive environment may be provided for experience by a user. Sensor data associated with the user may be received. The sensor data may be analyzed using at least one machine learning model to determine changes to a user state. Based at least in part upon the analyzed sensor data, modifications to be made to the virtual bioadaptive environment may be determined. A modified virtual bioadaptive environment may be provided to the user.

### CROSS REFERENCE TO RELATED APPLICATIONS

This application is a 371 National Phase application from PCT Application No. PCT/US22/50755, filed on Nov. 22, 2022, and entitled “APPARATUSES, SYSTEMS, AND METHODS FOR A REAL TIME BIOADAPTIVE STIMULUS ENVIRONMENT,” which claims priority to U.S. Provisional Patent Application No. 63/282,635 filed Nov. 23, 2021, and entitled “APPARATUSES, SYSTEMS, AND METHODS FOR A REAL TIME BIOADAPTIVE STIMULUS ENVIRONMENT,” which is hereby incorporated by reference herein in its entirety for all purposes.

### BACKGROUND

Since the advent of modern medicine, many treatments and drug schedules have been developed. However, while many such treatments may be effective, the outset of some treatments may be uncomfortable for some patients. For example, in the case of treatment via psychedelics, many patients have never had a psychedelic experience and may find their initial experience to be rather intense.

Preparing a patient for an upcoming treatment may be beneficial prior to administration of a drug. Collected patient data can be analyzed to help assess how a patient may be feeling at a given point in time. Further, collected data may be processed, using deep learning and machine learning techniques, to help tailor the patient's experience both during and after dosing.

### BRIEF DESCRIPTION OF THE DRAWINGS

Various embodiments in accordance with the present disclosure will be described with reference to the drawings, in which:

FIGS. 1A and 1B illustrate example scene depictions that can be utilized in accordance with one or more embodiments.

FIGS. 2A and 2B illustrate example sensor data that can be utilized in accordance with one or more embodiments.

FIG. 3 illustrates an example system that can be utilized to implement one or more aspects of the various embodiments.

FIG. 4 illustrates an example method that can be utilized to implement one or more aspects of the various embodiments.

FIG. 5 illustrates an example of an environment that can be utilized to implement one or more aspects of the various embodiments.

FIG. 6 illustrates an example of an environment for implementing one or more aspects of the various embodiments.

FIG. 7 illustrates an example block diagram of an electronic device that can be utilized to implement one or more aspects of the various embodiments.

FIG. 8 illustrates components of another example environment in which aspects of various embodiments can be implemented.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

A real time bioadaptive stimulus environment with audio, visual, and olfactory components to enhance psychedelic therapy is provided. A system associated with the environment may be configured to provide a real time bioadaptive sensory environment that tailors the psychedelic experience to the temporal evolution of the patient's subjective state in a dosing session using low latency and temporally synchronized objective psychophysiological measurements. A biosensor suite associated with the environment may record measurements and tailor audio, visual, and olfactory stimuli to a patient's subjective state as it evolves, to promote a positive psychedelic experience that may lead to desired therapeutic outcomes. In accordance with an example embodiment, the stimuli may be tailored using artificial intelligence and machine learning techniques.

The system may also provide direct visual, olfactory, and auditory stimuli to the patient that is procedurally generated in real time to help guide each patient through a unique psychedelic experience. For example, the psychedelic experience may be tailored and/or controlled to produce desired treatment outcomes. As a further example, if a machine learning model has determined that the patient is uncomfortable and would benefit from listening to a soothing sound, the system may play such a soothing sound for the patient to listen to. In at least this example, the system may operate in real time, and the patient's reaction to the soothing sound can be measured and interpreted by the system, enabling the system to further alter the sound output. Sound output may be altered in volume, and can also be altered to play different kinds of sounds such as a melody, a single tone, or a combination of tones.

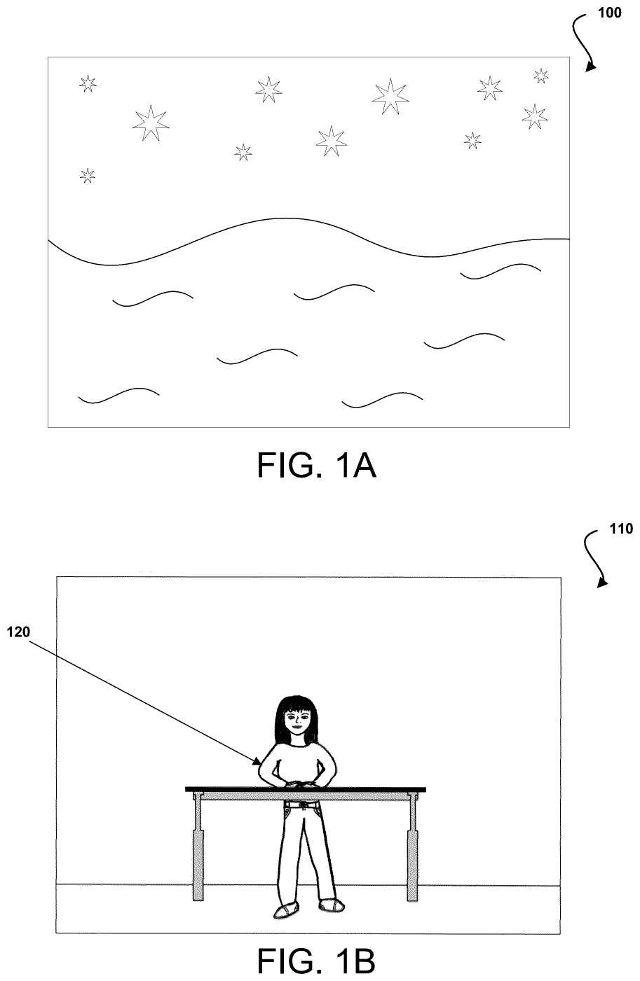

FIGS. 1A and 1B illustrate example scene depictions 100, 110 that can be utilized in accordance with one or more embodiments. In accordance with an example embodiment, a virtual reality (VR) enabled setting may be utilized for preparation, dosing, and integration sessions in order to help augment the psychedelic experience. For example, the VR setting may include either or both of an environmental setting, such as the scene depiction 100, and an avatar guide 120 as shown in scene depiction 110. While this example refers to the use of VR, augmented reality or enhanced reality may also be utilized in accordance with various embodiments. In an environmental setting, in accordance with an example embodiment, the patient may be able to experience a three-dimensional world and environment (for example, a forest or beach), while in an avatar guide, the patient may interact with a persona that guides them through the experience. The system may utilize an environmental setting, an avatar guide setting, and/or a combination of both settings. In an example embodiment, augmentation may be utilized for both preparation of the experience and/or post-experience review. In this example, the system may record which stimuli were presented and the time at which they were presented, allowing for an experience “playback.” Further, the system may record and store audio and/or visual outputs associated with the patient, such that the therapist or patient may later watch the patient's experience from a different perspective, such as a perspective view of the patient. In some example embodiments, the perspective view and the stimuli schedule of the session can be superimposed or overlaid such that, upon review of the session, the reviewer can observe the patient while also reading the stimuli.

In at least one example embodiment, the system may include a patient virtual profile. The patient virtual profile may be created with a priori settings that may then be modified based on patient experiences. The settings in the patient virtual profile may be initially populated when the user first registers. For example, the system may inquire about various personal details at registration. In another example, the therapist, administrator, or other user may determine the settings. The patient may then interact with the system to prepare for a session, such as a dosing session, by experiencing a preparatory experience, such as a simulated psychedelic experience, with immersive video and audio stimuli, such as that provided by a VR headset. In accordance with an example embodiment, psychophysiological patient responses may be monitored and recorded. In this example, recorded responses may be utilized to calibrate the dosing session and to build a posterior profile of the optimal experience settings. As a non-limiting example, the experience may modulate the environment of the person, the music they listen to, or the story the patient is exposed to in the VR environment. These changes may be used to modify the set and setting of the psychedelic experience and, therefore, guide the patient toward a specific emotional response such as calmness, excitement, or curiosity. As each person responds to different stimuli differently, the calibration may be used to learn how an individual responds to different sensory input. In an alternative embodiment, the responses may be used to calibrate the system for any type of treatment and/or session.

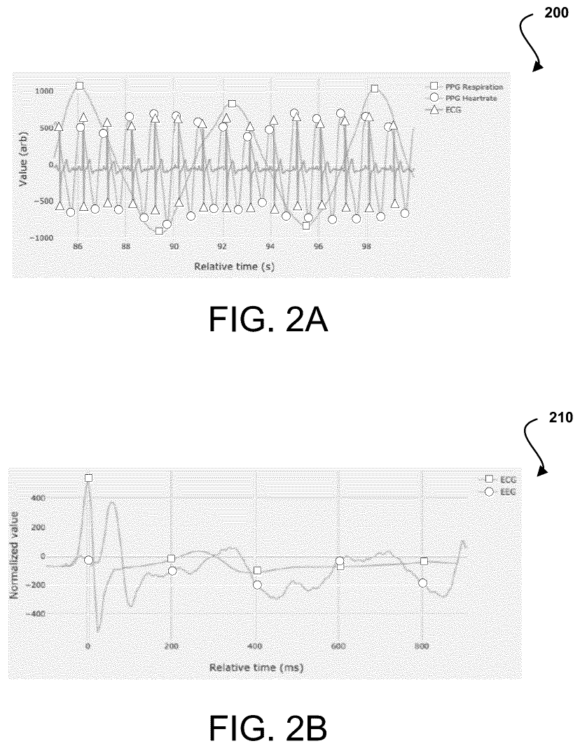

FIGS. 2A and 2B illustrate example sensor data 200, 210 that can be utilized in accordance with one or more embodiments. In accordance with an example embodiment, the system can be configured to determine photoplethysmogram (PPG) respiration, PPG heart rate, electrocardiogram (ECG) data, and electroencephalogram (EEG) data, among other such measurements. The system can further be configured to determine electric activity of the brain, such as by using an EEG sensor. An EEG sensor may include, but is not limited to, a four-channel headband measuring the left and right temporal parietal areas and the left and right anterior-frontal areas.

During dosing, a patient may also be monitored through a variety of hardware systems and sensors including, but not limited to, a camera (including, but not limited to, a high-definition calibrated camera array that can monitor heart rate/pulse, breathing, body temperature, flush response, facial expression, and/or pupil response); a microphone (including, but not limited to, a beamforming microphone array to capture spatially localized audio); electroencephalography; wearables (including, but not limited to, a wrist-based electromyography wearable and an electrocardiogram chest strap); and other suitable hardware components.

In accordance with an example embodiment, the system may include a computer in the same vicinity as the sensors, configured to record and process temporally synchronous signals locally. In this example, the recorded signals and/or results may be uploaded to cloud infrastructure for later post-processing. A machine learning model, such as an at-the-edge machine learning model, may be trained to simultaneously process the various information to tailor the psychedelic experience through modulation of the visual, audio, and olfactory stimulus environment presented to the patient. In an example embodiment, the system may change the immersive visual environment that the patient experiences. Using the analyzed sensor data, the system may determine, using machine learning, which environmental changes to make such that the changes may be automatically made in real time or near-real time. If, for example, a given data point in the sensor data falls below a determined threshold or score, the system may determine that corrective action is needed. Depending on the specific data point, the system may decide which stimulus or stimuli to adjust. For example, visual representations of a beach may be provided for presentation instead of a library or a river. Further, the accompanying audio may also be changed. For example, classical music may be changed to jazz, or exciting music may be changed to calming music. The olfactory stimulus may also change. For example, the refreshing smell of a river may be provided compared to the musty smell of a library. Further, audio pitch, audio volume, scene brightness, avatar type, scent type, or scent strength may be manually or automatically adjusted, among other such options. In accordance with an example embodiment, continuous psychophysiological data may be taken from a patient to further modulate the adaptive stimulus environment, as well as to update the machine learning models that may be guiding the session.

After dosing, a patient may be able to recreate part of their psychedelic experience through replaying the same sensory stimuli they experienced during dosing, as appropriate under supervision, to assist with integration. A group integration experience may be held virtually as appropriate and depending on the treatment profile. For example, multiple patients may interact with multiple biosensor suites, or multiple patients may interact with the same biosensor suite.

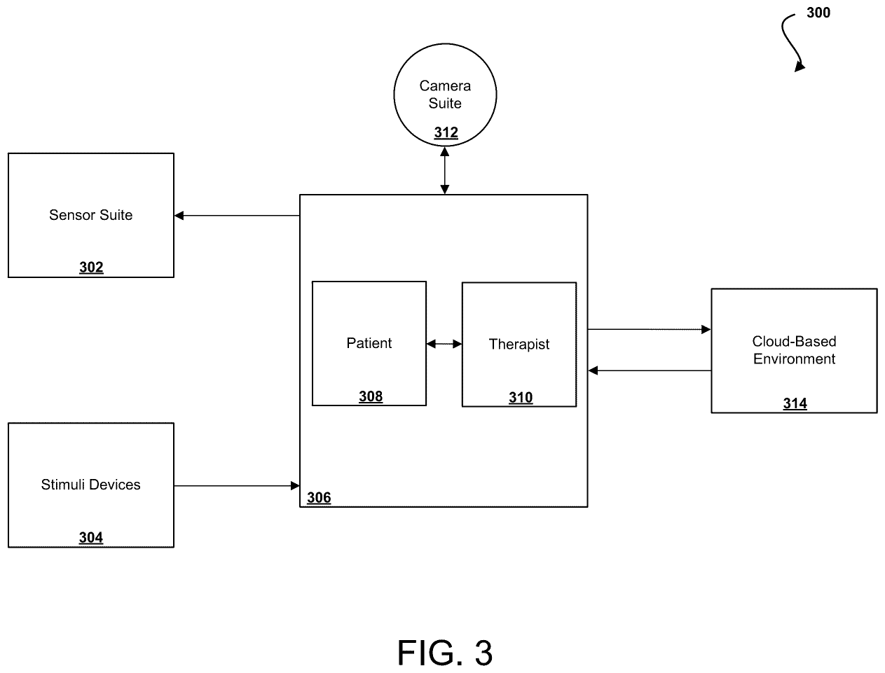

FIG. 3 illustrates an example system 300 that can be utilized to implement one or more aspects of the various embodiments. In accordance with an example embodiment, the system 300 may aid the entire therapeutic process. For example, the system 300 may be able to help patients during the preparatory stage, dosing stage, and integration stage. Moreover, the system may enhance the safety, efficacy, and accessibility profile of treatment, such as by better regulating the experience and/or deploying it at a greater scale.

In accordance with an example embodiment, the system 300 may include a sensor suite 302 comprising associated sensor data. The sensor suite 302 may provide for real time monitoring of a patient 308 and/or therapist 310 within a therapy environment 306. In an example embodiment, the therapist 310 may have a subset of sensors that are either visible or not visible to the patient 308. For example, electromyography (EMG) may be used to help analyze nerve-to-muscle signal transmission. Sensors specific to the therapist 310 may be configured so as to not impact the patient experience. In at least one example embodiment, a camera suite 312 may also be utilized to help collect visual data for the patient 308 and/or therapist 310. Sensor suite devices may synchronize wirelessly to a compact server such as a single-board computer. In accordance with an example embodiment, one or more devices of the sensor suite 302 may be synchronized via a wired or wireless connection. A machine learning model may be employed to facilitate the adjustment of visual, audio, and/or olfactory stimuli using one or more stimuli devices 304. Adjustment of the various stimuli may be facilitated, at least in part, using a virtual reality device, an augmented reality device, an enhanced reality device, or any other such presentation device. Further, stimuli may be adjusted using a combination of devices, such as a speaker with audio output or devices which may emit a variety of scents. During or after monitoring, data may be relayed to a cloud-based environment 314 for further analysis. Analysis may be performed by using machine learning, for example. Over the course of monitoring, or after monitoring, a therapist may be able to review biofeedback processes on an electronic device, including but not limited to a smart phone, personal computer, or tablet. The system may also maintain and/or facilitate cloud-based infrastructure that allows various bioadaptive enabled software to be distributed and accessed from a centralized repository. Such a system may allow continuous virtual software updates for a pre-determined hardware profile.

The system may be used for a number of applications. For example, the system may provide an at-the-edge biosensor suite that objectively measures patient psychophysiological biomarkers including, but not limited to, electroencephalography, pulse, electrocardiogram, facial expression and flush responses, pupil response, muscle tenseness, and/or electrodermal response for both immediate processing and long term cloud enabled storage. Data correlating to individual metrics (e.g., pulse, facial expression, flush response, etc.) may be stored locally and/or in cloud storage. Data may be stored in a raw or processed format. In various embodiments, the analysis of the sensor information to identify biomarkers and provide continuous feedback may be done in the cloud or at-the-edge depending on the complexity of the model.

The sensor suite 302 may include, but is not limited to, electroencephalography (EEG), electrocardiography (ECG), photoplethysmography, pulse oximetry, electromyography (EMG), spatialized audio recording, and/or a camera array. The camera array may include high-resolution color (RGB), thermal, and/or depth sensors with adequate frame rates to measure in real-time: pulse, respiration, body temperature changes, facial flush responses, facial expressions, pupillometry, eye movements, and general bodily movements, such as movements that may indicate physical agitation. Sensors may also be embedded in hardware, such as motion sensors in a VR headset.

In accordance with an example embodiment, the sensor suite 302 may enable various flows of communication. In accordance with a non-limiting example, communication may include a person-to-person interaction, information flow of data from person to device, or stimulus from device to person. In an example embodiment, the sensor suite 302 may be configured to receive data from the patient 308. Stimulus hardware (e.g., visual, auditory, and olfactometry) may be configured to deliver immersive stimuli 304 to the patient 308. The patient 308 and therapist 310 may be in person-to-person communication. The camera suite 312 may include one or more microphone arrays, and may be configured to receive data from the patient 308 and/or therapist 310. The patient 308, therapist 310, camera suite 312, and other sensor devices may be in two-way communication with the cloud-based environment 314. In various embodiments, any of the components may be configured to communicate with any of the other components and/or parties.

The system, in accordance with an example embodiment, may include a login portal configured for anonymity during clinical trial settings. Further, the system may be configured for automated cross-platform or cross-operating system digital biomarker and sensor data collection, that may be integrated into a single cloud-native database. The system may also provide a synchronized content database that can be remotely updated for new studies and patients.

In accordance with an example embodiment, a front-end application may be used across all operating systems, mobile devices, and personal computers. The front-end application may allow for easier validation of the application for regulatory purposes as one codebase. The application may allow for the display of custom biomarkers to a care team and patient, as well as custom alerting based on collected data. The application may allow or “full circle” machine learning because the system may be able to collect data, upload data, analyze data, identify triggers, and send information back to the care team.

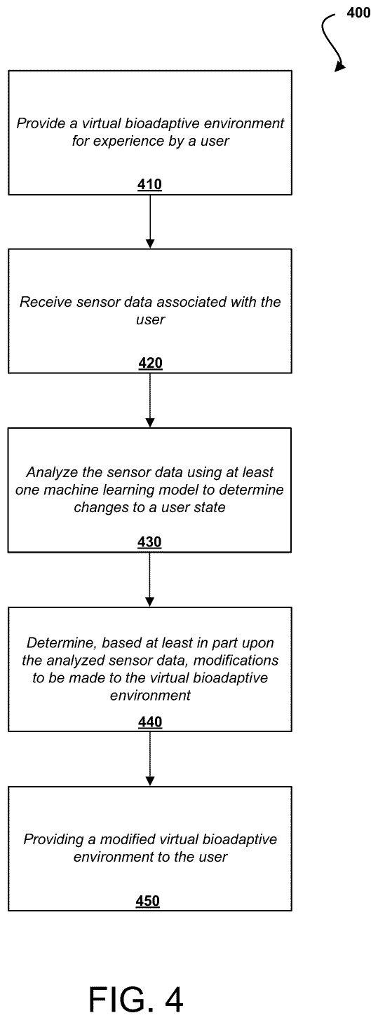

FIG. 4 illustrates an example method 400 that can be utilized to implement one or more aspects of the various embodiments. It should be understood that for any process herein there can be additional, fewer, or alternative steps performed in similar or alternative orders, or in parallel, within the scope of the various embodiments unless otherwise specifically stated. In accordance with an example embodiment, a virtual bioadaptive environment may be provided for experience by a user 410. The virtual bioadaptive environment may be provided, in at least some embodiments, on a virtual reality device, an augmented reality device, an enhanced reality device, or any other such presentation device(s) or system(s). Sensor data associated with the user may be received 420, such as through the presentation device(s) or system(s). The sensor data may be analyzed using at least one machine learning model to determine one or more changes to a user state 430. For example, it can be determined that at least a subset of the sensor data falls below a determined threshold level. Based at least in part upon the analyzed sensor data, one or more modifications to be made to the virtual bioadaptive environment can be determined 440. A modified virtual bioadaptive environment may be provided on the presentation device(s) or system(s) 450.

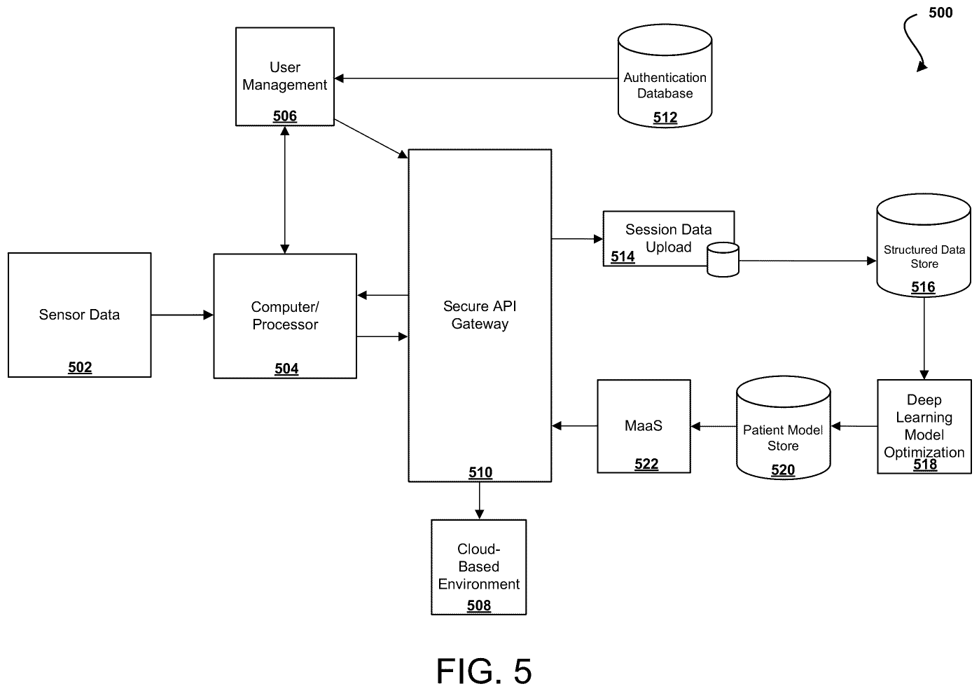

FIG. 5 illustrates an example of an environment 500 that can be utilized to implement one or more aspects of the various embodiments. In accordance with an example embodiment, the environment may be a computational layer. A sensor suite may provide sensor data 502 and be in communication with a computer or processor 504. The computer 504 may be in communication with a user management node 506, such that a therapist, patient, or other party may be authenticated. In accordance with an example embodiment, a patient may subscribe to the system's digital infrastructure when they are prescribed a treatment. Upon registering, a unique identifier may be assigned to the patient. The unique identifier may be used for future tracking and potential integration with other components of the system or tertiary systems, such as a companion application. The unique identifier may be used as, or in conjunction with, metadata, which may be tagged on data correlated to that patient. For example, video files collected by a camera array or audio files collected by a microphone may be correlated with a unique identifier and metadata. Prior to dosing, a patient may be able to review settings, as may be appropriate, through the system infrastructure (e.g., a mobile application, a web portal, or suitable alternative) to fully prepare for the dosing experience.

The computer 504 may also be in communication with a cloud-based environment 508 via a secure application programming interface (API) gateway 510. The cloud-based environment 508 may include or be in communication with an authentication database 512, where the authentication database 512 is in communication with the user management node 506 and is configured to help manage users. The cloud-based environment 508 may include a session data upload 514 that may extract, transform, and load data to a structured data store 516. The structured data store 516 may be in communication with a deep learning or machine learning optimization node 518. The deep learning or machine learning optimization node 518 may be configured to analyze both a priori and per patient (posterior model) data to create an optimal experience that is uniquely tailored to the patient. In an example embodiment, the deep learning or machine learning model optimization node may receive information about the global population, as well as information containing patient unique or specific preferences.

As a non-limiting example, if the sound of the ocean is universally calming and the patient prefers jazz music to classical music to relax, the deep learning or machine learning model optimization node may create or generate a custom experience combining both the sound of the ocean and the genre of jazz. Further, the deep learning or machine learning model optimization node may also modulate the experience, such as by selecting a specific scene or music. In such an embodiment, each specific image or audio file may have varying intensities of various traits. For example, a first beach scene may be exceptionally calming, while a second beach scene may be mildly calming. Accordingly, in such an example, the deep learning or machine learning model optimization node may discriminate between specific instances of each class of stimuli and present each specific instance based on global population information and/or patient-specific information. A patient model store 520 may then receive optimized data from the deep learning or machine learning model optimization node. In an example embodiment, the patient model store may be in communication with a “model as a service,” (MaaS) 522 so as to provide real time on-demand models per patient. In order to provide models per patient, the MaaS may be in communication with the computer 504 via the secure API gateway 510.

A system associated with the environment may utilize a variety of deep learning, general machine learning, and/or statistical models, where the models are evaluated for optimal performance. For example, the system may employ any number of or combination of the following models: perceptron, feed forward, radial basis network, deep feed forward, recurrent neural network, long/short term memory, gated recurrent unit, auto encoder, variational auto encoder, sparse auto encoder, denoising auto encoder, Markus chain, Hopfield network, Boltzman machine, restricted Boltzman machine, deep believe network, deep convolutional network, deep network, deep convolutional inverse graphics network, generative adversarial network, liquid state machine, extreme learning machine, echo network machine, Kohonen network, deep residual network, support vector machine, and neural Turing machine.

The system may include a computational at-the-edge layer. The sensor suite may be synchronized against a computer or computing device during a dosing session. The computer may be pre-loaded with a global machine learning model that can be overwritten or updated from cloud-based network settings. The global machine learning model may not account for patient preferences and may be used if no prior information from the patient is available or needed. The patient-specific machine learning model may incorporate information from the patient (either real time or during training) via the aforementioned deep learning or machine learning model optimization node. The model may execute in real time and provide feedback to devices for the patient and/or therapist. The user profile may be downloaded from the cloud with additional model parameters, as may be necessary. User biomarkers and sensor data, for either or both the patient and therapist, may be uploaded back to the cloud-based network for further updates and evaluation. In some example embodiments, the system may develop a continuous feedback system, allowing the administrator, user, or medical professional to improve a baseline psychedelic experience and to develop a unique patient specific profile that may be used for subsequent dosing sessions. For example, a patient-specific profile may be stored in a cloud-based environment and may contain defaults or baselines to be utilized by the system for the patient's next session. In this example, by utilizing past data, the system may decrease the amount of time necessary to effectively adapt a patient to medication. Further, the system may also predict and adjust future baselines or defaults based on the current profile and past data.

The system may include and/or abide by a patient therapeutic timeline. The preparation step may include providing a pre-dosing “psychedelic experience” to prepare patients for a dosing session. The preparation step may also include measuring a patient response to pre-dosing to create a patient-specific digital experience. Further, in this pre-dosing step, the system may be configured to identify and/or flag any early warning signs and attempt to mitigate any potential issues.

The dosing step may include providing a customized digital experience by modulating visual, olfactory, and auditory stimuli. In this example embodiment, a real time biofeedback loop may use an at-the-edge model to modulate experiences to increase patient safety and improve therapeutic delivery. Moreover, in the dosing step, the system may record sensor information for the integration session and for model updates. In accordance with an example embodiment, the dosing session may involve administering some form of psilocybin to the patient.

An integration step may include activating memory of the experience by replaying the sensory feedback experienced during a dosage session, such as audio, olfactory, and visual. The integration step may allow in-group integration through VR infrastructure. Further, multiple self-guided integration sessions may be possible in accordance with an example embodiment. In an embodiment, the system may update both global and patient-specific models for future dosing sessions.

During dosing, the environment may be used and adjusted to modulate both the intensity and emotional valence of the experience based directly upon patient feedback as well as broader population-level data. In a further embodiment, the system may improve the integration session by giving the patient a way to relive any portion of their experience for further reflection. Such an environment may be used throughout the patient journey to further enhance the therapeutic model.

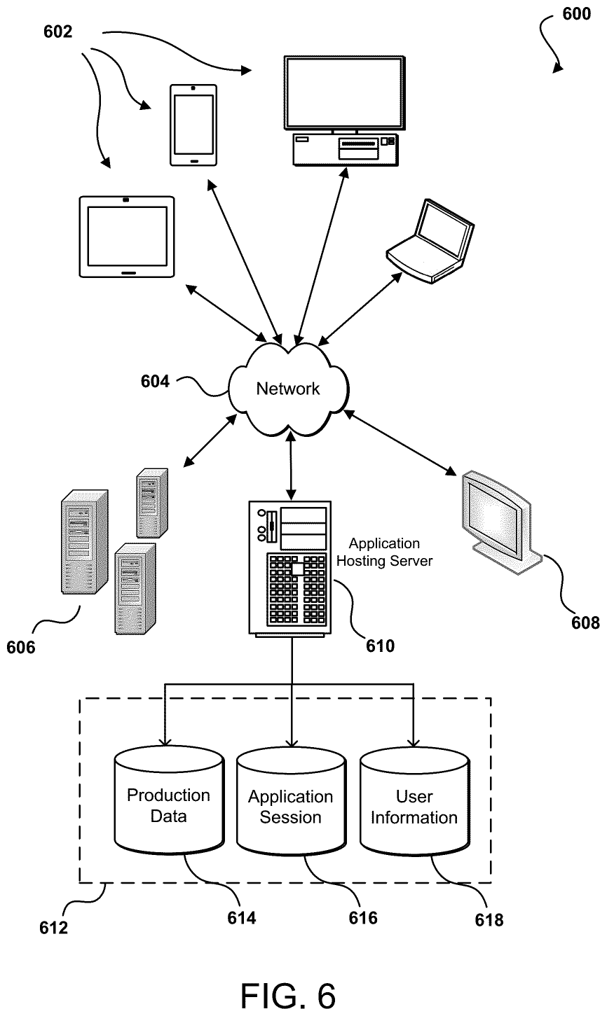

As discussed, different approaches can be implemented in various environments in accordance with the described embodiments. For example, FIG. 6 illustrates an example of an environment 600 for implementing one or more aspects of the various embodiments. As will be appreciated, although a Web-based environment is used for purposes of explanation, different environments may be used, as appropriate, to implement various embodiments. The system includes an electronic client device 602, 608, which can include any appropriate device operable to send and receive requests, messages or information over an appropriate network 604 and convey information back to a user of the device. Examples of such client devices include personal computers, virtual reality device(s), augmented reality device(s), enhanced reality device(s), cell phones, handheld messaging devices, laptop computers, set-top boxes, personal data assistants, electronic book readers and the like. The network can include any appropriate network, including an intranet, the Internet, a cellular network, a local area network or any other such network or combination thereof. Components used for such a system can depend at least in part upon the type of network and/or environment selected. Protocols and components for communicating via such a network are well known and will not be discussed herein in detail. Communication over the network can be enabled via wired or wireless connections and combinations thereof. In this example, the network includes the Internet, as the environment includes one or more servers 606 for receiving requests and serving content in response thereto, although for other networks, an alternative device serving a similar purpose could be used, as would be apparent to one of ordinary skill in the art.

The illustrative environment includes at least one application server 610 and a data store 612. It should be understood that there can be several application servers, layers or other elements, processes or components, which may be chained or otherwise configured, which can interact to perform tasks such as obtaining data from an appropriate data store. As used herein, the term “data store” refers to any device or combination of devices capable of storing, accessing and retrieving data, which may include any combination and number of data servers, databases, data storage devices and data storage media, in any standard, distributed or clustered environment. The application server 610 can include any appropriate hardware and software for integrating with the data store 612 as needed to execute aspects of one or more applications for the client device and handling a majority of the data access and business logic for an application. The application server provides access control services in cooperation with the data store and is able to generate content such as text, graphics, audio and/or video to be transferred to the user, which may be served to the user by the one or more servers 606, including a Web server, in the form of HTML, XML or another appropriate structured language in this example. The handling of all requests and responses, as well as the delivery of content between the client device 602, 608 and the application server 610, can be handled by the Web server of servers 606. It should be understood that the Web and application servers are not required and are merely example components, as structured code discussed herein can be executed on any appropriate device or host machine as discussed elsewhere herein.

The data store 612 can include several separate data tables, databases or other data storage mechanisms and media for storing data relating to a particular aspect. For example, the data store illustrated includes mechanisms for storing production data 614 and user information 618, which can be used to serve content for the production side. The data store is also shown to include a mechanism for storing log or application session data 616. It should be understood that there can be many other aspects that may need to be stored in the data store, such as page image information and access rights information, which can be stored in any of the above listed mechanisms as appropriate or in additional mechanisms in the data store 612. The data store 612 is operable, through logic associated therewith, to receive instructions from the application server 610 and obtain, update or otherwise process data in response thereto. In one example, a user might submit a request for transcribing, tagging, and/or labeling a media file. In this case, the data store might access the user information to verify the identity of the user and can provide a transcript including tags and/or labels along with analytics associated with the media file. The information can then be returned to the user, such as in a results listing on a Web page that the user is able to view via a browser on the user device 602, 608. Information for a particular feature of interest can be viewed in a dedicated page or window of the browser.

Each server typically will include an operating system that provides executable program instructions for the general administration and operation of that server and typically will include computer-readable medium storing instructions that, when executed by a processor of the server, allow the server to perform its intended functions. Suitable implementations for the operating system and general functionality of the servers are known or commercially available and are readily implemented by persons having ordinary skill in the art, particularly in light of the disclosure herein.

The environment in one embodiment is a distributed computing environment utilizing several computer systems and components that are interconnected via communication links, using one or more computer networks or direct connections. However, it will be appreciated by those of ordinary skill in the art that such a system could operate equally well in a system having fewer or a greater number of components than are illustrated in FIG. 6. Thus, the depiction of the system 600 in FIG. 6 should be taken as being illustrative in nature and not limiting to the scope of the disclosure.

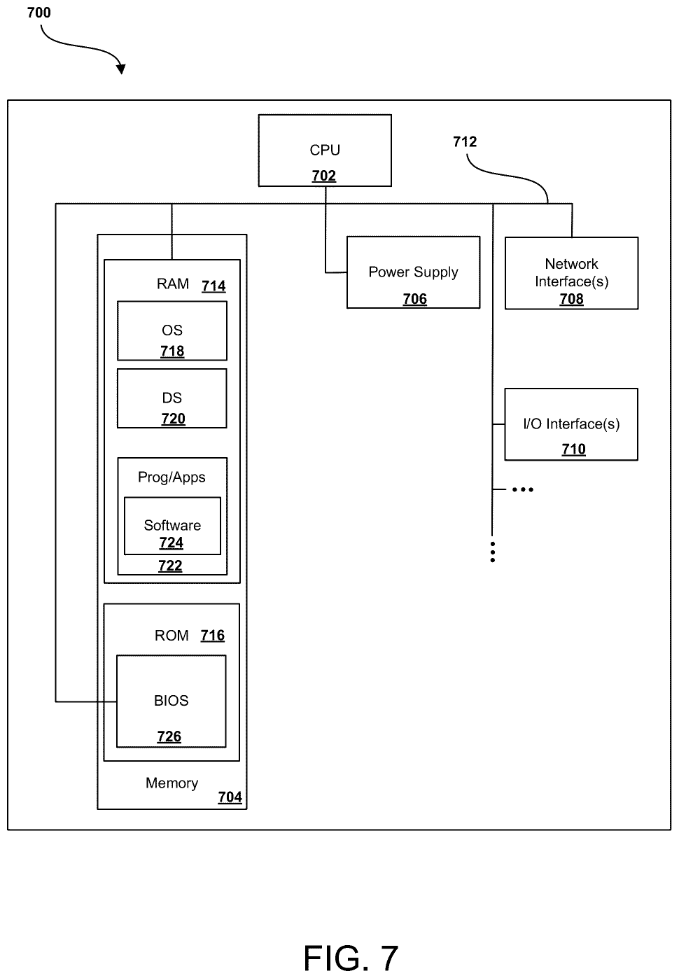

FIG. 7 illustrates an example block diagram of an electronic device that can be utilized to implement one or more aspects of the various embodiments. Instances of the electronic device 700 may include one or more servers and one or more client devices. In general, the electronic device may include a processor/CPU 702, memory 704, a power supply 706, and input/output (I/O) components/devices 710, e.g., microphones, speakers, displays, touchscreens, keyboards, mice, keypads, microscopes, GPS components, cameras, heart rate sensors, light sensors, accelerometers, targeted biometric sensors, neck wearables detecting brain activity, etc., which may be operable, for example, to provide graphical user interfaces or text user interfaces.

A user may provide input via a touchscreen of an electronic device 700. A touchscreen may determine whether a user is providing input by, for example, determining whether the user the user is touching the touchscreen with a part of the user's body such as their fingers. The electronic device 700 can also include a communications bus 712 that connects to the aforementioned elements of the electronic device 700. Network interfaces 708 can include a receiver and a transmitter (or a transceiver), and one or more antennas for wireless communications.

The processor 702 can include one or more of any type of processing device, e.g., a Central Processing Unit (CPU), and a Graphics Processing Unit (GPU). Also, for example, the processor can utilize central processing logic, or other logic, may include hardware, firmware, software or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 704, which can include Random Access Memory (RAM) 714 and Read Only Memory (ROM) 716, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 718, data storage 720, which may include one or more databases, and programs and/or applications 722, which can include, for example, software aspects of the program 724. The ROM 716 can also include Basic Input/Output System (BIOS) 726 of the electronic device 700.

Software aspects of the program 722 are intended to broadly include or represent all programming, applications, algorithms, models, software and other tools necessary to implement or facilitate methods and systems according to embodiments of the invention. The elements may exist on a single computer or be distributed among multiple computers, servers, devices, or entities.

The power supply 706 may contain one or more power components, and may help facilitate supply and management of power to the electronic device 700.

The input/output components, including Input/Output (I/O) interfaces 710, can include, for example, any interfaces for facilitating communication between any components of the electronic device 700, components of external devices, and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 710 and the bus 712 can facilitate communication between components of the electronic device 700, and in an example can ease processing performed by the processor 702.

Where the electronic device 700 is a server, it can include a computing device that can be capable of sending or receiving signals, e.g., a wired or wireless network, or may be capable of processing or storing signals, e.g., in memory as physical memory states. The server may be an application server that includes a configuration to provide one or more applications via a network to another device. Also, an application server may, for example, host a website that can provide a user interface for administration of example embodiments.

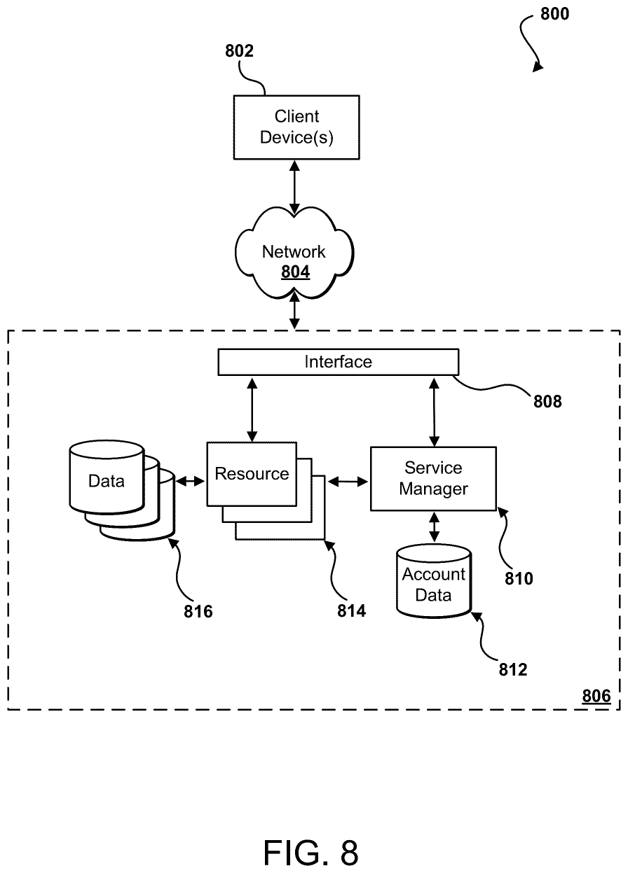

FIG. 8 illustrates an example environment 800 in which aspects of the various embodiments can be implemented. In this example a user is able to utilize one or more client devices 802 to submit requests across at least one network 804 to a multi-tenant resource provider environment 806. The client device can include any appropriate electronic device operable to send and receive requests, messages, or other such information over an appropriate network and convey information back to a user of the device. Examples of such client devices include personal computers, virtual reality device(s), augmented reality device(s), enhanced reality device(s), tablet computers, smart phones, notebook computers, and the like. The at least one network 804 can include any appropriate network, including an intranet, the Internet, a cellular network, a local area network (LAN), or any other such network or combination, and communication over the network can be enabled via wired and/or wireless connections. The resource provider environment 806 can include any appropriate components for receiving requests and returning information or performing actions in response to those requests. As an example, the provider environment might include Web servers and/or application servers for receiving and processing requests, then returning data, Web pages, video, audio, or other such content or information in response to the request.

In various embodiments, the provider environment may include various types of resources that can be utilized by multiple users for a variety of different purposes. As used herein, computing and other electronic resources utilized in a network environment can be referred to as “network resources.” These can include, for example, servers, databases, load balancers, routers, and the like, which can perform tasks such as to receive, transmit, and/or process data and/or executable instructions. In at least some embodiments, all or a portion of a given resource or set of resources might be allocated to a particular user or allocated for a particular task, for at least a determined period of time. The sharing of these multi-tenant resources from a provider environment is often referred to as resource sharing, Web services, or “cloud computing,” among other such terms and depending upon the specific environment and/or implementation. In this example the provider environment includes a plurality of resources 814 of one or more types. These types can include, for example, application servers operable to process instructions provided by a user or database servers operable to process data stored in one or more data stores 816 in response to a user request. As known for such purposes, the user can also reserve at least a portion of the data storage in a given data store. Methods for enabling a user to reserve various resources and resource instances are well known in the art, such that detailed description of the entire process, and explanation of all possible components, will not be discussed in detail herein.

In at least some embodiments, a user wanting to utilize a portion of the resources 814 can submit a request that is received to an interface layer 808 of the provider environment 806. The interface layer can include application programming interfaces (APIs) or other exposed interfaces enabling a user to submit requests to the provider environment. The interface layer 808 in this example can also include other components as well, such as at least one Web server, routing components, load balancers, and the like. When a request to provision a resource is received to the interface layer 808, information for the request can be directed to a service manager 810 or other such system, service, or component configured to manage user accounts and information, resource provisioning and usage, and other such aspects. A service manager 810 receiving the request can perform tasks such as to authenticate an identity of the user submitting the request, as well as to determine whether that user has an existing account with the resource provider, where the account data may be stored in at least one data store 812 in the provider environment. A user can provide any of various types of credentials in order to authenticate an identity of the user to the provider. These credentials can include, for example, a username and password pair, biometric data, a digital signature, a QR-based credential, or other such information.

The provider can validate this information against information stored for the user. If the user has an account with the appropriate permissions, status, etc., the resource manager can determine whether there are adequate resources available to suit the user's request, and if so can provision the resources or otherwise grant access to the corresponding portion of those resources for use by the user for an amount specified by the request. This amount can include, for example, capacity to process a single request or perform a single task, a specified period of time, or a recurring/renewable period, among other such values. If the user does not have a valid account with the provider, the user account does not enable access to the type of resources specified in the request, or another such reason is preventing the user from obtaining access to such resources, a communication can be sent to the user to enable the user to create or modify an account, or change the resources specified in the request, among other such options. In at least some example embodiments, a user may be authenticated to access an entire fleet of services provided within a service provider environment. In other example embodiments, a user's access may be restricted to specific services within the service provider environment using one or more access policies tied to the user's credential(s).

Once the user is authenticated, the account verified, and the resources allocated, the user can utilize the allocated resource(s) for the specified capacity, amount of data transfer, period of time, or other such value. In at least some embodiments, a user might provide a session token or other such credentials with subsequent requests in order to enable those requests to be processed on that user session. The user can receive a resource identifier, specific address, or other such information that can enable the client device 802 to communicate with an allocated resource without having to communicate with the service manager 810, at least until such time as a relevant aspect of the user account changes, the user is no longer granted access to the resource, or another such aspect changes.

The service manager 810 (or another such system or service) in this example can also function as a virtual layer of hardware and software components that handles control functions in addition to management actions, as may include provisioning, scaling, replication, etc. The resource manager can utilize dedicated APIs in the interface layer 808, where each API can be provided to receive requests for at least one specific action to be performed with respect to the data environment, such as to provision, scale, clone, or hibernate an instance. Upon receiving a request to one of the APIs, a Web services portion of the interface layer can parse or otherwise analyze the request to determine the steps or actions needed to act on or process the call. For example, a Web service call might be received that includes a request to create a data repository.

An interface layer 808 in at least one embodiment includes a scalable set of user-facing servers that can provide the various APIs and return the appropriate responses based on the API specifications. The interface layer also can include at least one API service layer that in one embodiment consists of stateless, replicated servers which process the externally-facing user APIs. The interface layer can be responsible for Web service front end features such as authenticating users based on credentials, authorizing the user, throttling user requests to the API servers, validating user input, and marshalling or unmarshalling requests and responses. The API layer also can be responsible for reading and writing database configuration data to/from the administration data store, in response to the API calls. In many embodiments, the Web services layer and/or API service layer will be the only externally visible component, or the only component that is visible to, and accessible by, users of the control service. The servers of the Web services layer can be stateless and scaled horizontally as known in the art. API servers, as well as the persistent data store, can be spread across multiple data centers in a region, for example, such that the servers are resilient to single data center failures.

The various embodiments can be further implemented in a wide variety of operating environments, which in some cases can include one or more user computers or computing devices which can be used to operate any of a number of applications. User or client devices can include any of a number of general purpose personal computers, such as desktop or laptop computers running a standard operating system, as well as cellular, wireless and handheld devices running mobile software and capable of supporting a number of networking and messaging protocols. Such a system can also include a number of workstations running any of a variety of commercially-available operating systems and other known applications for purposes such as development and database management. These devices can also include other electronic devices, such as dummy terminals, thin-clients, gaming systems and other devices capable of communicating via a network.

Most embodiments utilize at least one network that would be familiar to those skilled in the art for supporting communications using any of a variety of commercially available protocols, such as TCP/IP, FTP, UPnP, NFS, and CIFS. The network can be, for example, a local area network, a wide-area network, a virtual private network, the Internet, an intranet, an extranet, a public switched telephone network, an infrared network, a wireless network and any combination thereof. In embodiments utilizing a Web server, the Web server can run any of a variety of server or mid-tier applications, including HTTP servers, FTP servers, CGI servers, data servers, Java servers and business application servers. The server(s) may also be capable of executing programs or scripts in response requests from user devices, such as by executing one or more Web applications that may be implemented as one or more scripts or programs written in any programming language, such as Java®, C, C# or C++ or any scripting language, such as Perl, Python or TCL, as well as combinations thereof. The server(s) may also include database servers, including without limitation those commercially available from Oracle®, Microsoft®, Sybase® and IBM®.

The environment can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (SAN) familiar to those skilled in the art. Similarly, any necessary files for performing the functions attributed to the computers, servers or other network devices may be stored locally and/or remotely, as appropriate. Where a system includes computerized devices, each such device can include hardware elements that may be electrically coupled via a bus, the elements including, for example, at least one central processing unit (CPU), at least one input device (e.g., a mouse, keyboard, controller, touch-sensitive display element or keypad) and at least one output device (e.g., a display device, printer or speaker). Such a system may also include one or more storage devices, such as disk drives, optical storage devices and solid-state storage devices such as random access memory (RAM) or read-only memory (ROM), as well as removable media devices, memory cards, flash cards, etc. Such devices can also include a computer-readable storage media reader, a communications device (e.g., a modem, a network card (wireless or wired), an infrared communication device) and working memory as described above. The computer-readable storage media reader can be connected with, or configured to receive, a computer-readable storage medium representing remote, local, fixed and/or removable storage devices as well as storage media for temporarily and/or more permanently containing, storing, transmitting and retrieving computer-readable information.

The system and various devices also typically will include a number of software applications, modules, services or other elements located within at least one working memory device, including an operating system and application programs such as a client application or Web browser. It should be appreciated that alternate embodiments may have numerous variations from that described above. For example, customized hardware might also be used and/or particular elements might be implemented in hardware, software (including portable software, such as applets) or both. Further, connection to other computing devices such as network input/output devices may be employed. Storage media and other non-transitory computer readable media for containing code, or portions of code, can include any appropriate media known or used in the art, such as but not limited to volatile and non-volatile, removable and non-removable media implemented in any method or technology for storage of information such as computer readable instructions, data structures, program modules or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices or any other medium which can be used to store the desired information and which can be accessed by a system device. Based on the disclosure and teachings provided herein, a person of ordinary skill in the art will appreciate other ways and/or methods to implement the various embodiments.

The specification and drawings are, accordingly, to be regarded in an illustrative rather than a restrictive sense. It will, however, be evident that various modifications and changes may be made thereunto without departing from the broader spirit and scope of the invention as set forth in the claims.

What is claimed is:

1. A computer-implemented method, comprising:
providing, from a presentation device, a virtual bioadaptive environment for experience by a user;
receiving, from the presentation device, sensor data associated with the user;
analyzing the sensor data using a machine learning model to determine one or more changes to a user state;
determining, based at least in part upon the analyzed sensor data, one or more modifications to be made to the virtual bioadaptive environment; and
providing a modified virtual bioadaptive environment on the presentation device.

2. The computer-implemented method of claim 1, wherein the virtual bioadaptive environment includes at least one of audio stimuli, visual stimuli, and olfactory stimuli.

3. The computer-implemented method of claim 1, wherein the virtual bioadaptive environment is provided, at least in part, using virtual reality, augmented reality, or enhanced reality.

4. The computer-implemented method of claim 2, wherein the visual stimuli include at least one of scene imagery and an avatar guide.

5. The computer-implemented method of claim 1, wherein the virtual bioadaptive environment is changed automatically in real time or near-real time.

6. The computer-implemented method of claim 1, further comprising:
storing the changed virtual bioadaptive environment to a user profile specific to the user.

7. A non-transitory computer-readable medium storing instructions, which, when executed by at least one processor, cause the at least one processor to:
provide, from a presentation system, a virtual bioadaptive environment for experience by a user;
receive, from the presentation system, sensor data associated with the user;
analyze the sensor data using a machine learning model to determine one or more changes to a user state; and
provide a modified virtual bioadaptive environment on the presentation system based, at least in part, upon the analyzed sensor data.

8. The non-transitory computer-readable medium of claim 7, wherein the instructions, when executed by the at least one processor, cause the at least one processor to further:
determine, based at least in part upon the analyzed sensor data, that at least a subset of the sensor data is below a threshold level; and
provide the modified virtual bioadaptive environment based, at least in part, upon the subset of the sensor data being below the threshold level.

9. The non-transitory computer-readable medium of claim 7, wherein the virtual bioadaptive environment includes at least one of audio stimuli, visual stimuli, and olfactory stimuli.

10. The non-transitory computer-readable medium of claim 7, wherein the virtual bioadaptive environment is provided, at least in part, using virtual reality, augmented reality, or enhanced reality.

11. The non-transitory computer-readable medium of claim 9, wherein the visual stimuli include at least one of scene imagery and an avatar guide.

12. The non-transitory computer-readable medium of claim 7, wherein the virtual bioadaptive environment is changed automatically in real time or near-real time.

13. The non-transitory computer-readable medium of claim 7, wherein changing the virtual bioadaptive environment includes changing at least one of: audio type, audio pitch, audio volume, scene type, scene brightness, and scent.

14. A system, comprising:
a presentation device;
at least one processor; and
memory, the memory storing instructions which, when executed by the at least one processor, cause the at least one processor to:
provide, from the presentation device, a virtual bioadaptive environment for experience by a user;
receive, from the presentation device, sensor data associated with the user;
analyze the sensor data using a machine learning model to determine one or more changes to a user state; and
provide a modified virtual bioadaptive environment based, at least in part, upon the analyzed sensor data.

15. The system of claim 14, wherein the instructions which, when executed by the at least one processor, cause the at least one processor to further:
determine, based at least in part upon the analyzed sensor data, that at least a subset of the sensor data is below a threshold level; and
provide the modified virtual bioadaptive environment based, at least in part, upon the subset of the sensor data being below the threshold level.

16. The system of claim 14, wherein the virtual bioadaptive environment includes at least one of audio stimuli, visual stimuli, and olfactory stimuli.

17. The system of claim 14, wherein the virtual bioadaptive environment is provided, at least in part, using virtual reality, augmented reality, or enhanced reality.

18. The system of claim 16, wherein the visual stimuli include at least one of scene imagery and an avatar guide.

19. The system of claim 14, wherein the virtual bioadaptive environment is changed automatically in real time or near-real time.

20. The system of claim 14, wherein changing the virtual bioadaptive environment includes changing at least one of: audio type, audio pitch, audio volume, scene type, scene brightness, and scent.
